(19) United States
(12) Patent Application Publication
(10) Pub. No.: US 2026/0335655 A1
(43) Pub. Date: Oct. 29, 2026

(54) SYSTEMS AND METHODS FOR RELATIVE POSITIONING OF TRANSMITTING AND RECEIVING COILS IN IN-CANAL WIRELESS TRANSMISSION SYSTEM

(71) Applicant: SMART SOUND LTD., Nirit (IL)

(72) Inventor: Harel ZILBERSHLAG, Givat Shmuel (IL)

(21) Appl. No.: 19/249,309

(22) Filed: Jun. 25, 2025

Related U.S. Application Data
(60) Provisional application No. 63/795,473, filed on Apr. 27, 2025.

(51) Int. Cl. A61N 1/372; A61N 1/08; A61N 1/36

(52) U.S. Cl. CPC A61N 1/372; A61N 1/08; A61N 1/36038

(57) ABSTRACT

An in-canal positioning and orienting device is provided for assisting in placement of a receiving coil to be implanted within a human ear and/or a transmitting coil to be placed within an ear canal one in respect to the other. The in-canal positioning and orienting device comprising a transducer configured to collect feedback comprising features characterizing a positioning and orientation of the receiving coil to be implanted and the transmitting coil to be placed within the ear canal. The transmitting and the receiving coils are parts of a hidden cochlear in canal wireless transmission system.

### CROSS REFERENCE TO RELATED APPLICATION

This application claims priority to U.S. Provisional Patent Application Ser. No. 63/795,473, filed Apr. 27, 2025.

### FIELD OF THE INVENTION

The present disclosed subject matter relates to in-canal wireless transmission systems. More particularly, the present disclosed subject matter relates to methods and systems for accurate positioning of transmitting and receiving coils to achieve an efficient transmission therebetween.

### BACKGROUND OF THE INVENTION

A cochlear implant is a small and complex electronic device that provides a sense of sound to a person who is profoundly deaf or severely hard-of-hearing. The implant consists of an external portion that is usually mounted behind the ear and a second portion that is surgically placed under the skin. In a hidden cochlear implant, the external portion is placed within the ear canal, physically closer to the implanted portion. This technology, which is implemented in devices indicated as in-canal wireless transmission systems (ICWTS), are described in details in U.S. Pat. No. 11,291,839 entitled “HIDDEN COCHLEAR IMPLANT SYSTEM” and in U.S. patent application Ser. No. 19/191,047 entitled “ HIDDEN COCHLEAR IMPLANT SYSTEM WITH AN IN-CANAL WIRELESS TRANSMISSION” filed by the inventor of the present subject matter on Apr. 28, 2025. The external portion that is inserted within the ear canal comprises the key component of the device which is a transmitter coil. As a consequence, the shape and dimensions of the specific ear canal to within the external portion of the ICWTS is inserted, is to be considered.

In general, the ear canal has been characterized as a structure of approximately 2.5 cm in length with an average width of 0.6 cm. The ear canal has an “S” shaped structure when observing down from the top of the head. The orientation of the transmitter coil within this structure has a significant effect on the efficiency and functionality of the power that is transmitted within the ICWTS.

FIG. 1 illustrates a perspective view of an exemplary prior art hidden cochlear implanted system that is implanted near internal organs of a human ear. Only important elements are described and numbered herein to maintain simplicity.

A hidden cochlear implanted unit 1 comprises an implanted unit 15 implanted within a human ear 9 beneath the skin, and a canal unit 10 that is inserted within the ear canal 8. According to this embodiment, part of the implanted unit 15 is configured to be implanted in a cochlea 11 of a user's ear. The implanted unit 15 comprises a cochlear receiver coil 152 and an electrode array 156.

A canal unit 10 is inserted in the ear canal 8 of a user's ear 9. The canal unit 10 comprises a canal microphone 104 residing at a side of the canal unit 10 that faces outside ear canal 8 and can easily receive sound signals that enter into the ear canal from the outer environment. The inward side of the canal unit 10 points toward the ear drum 12 and the cochlea 11 and is provided with a canal transmitting coil 108. The transmitting coil 108 is adjacent to the implanted receiving coil 152; both are separated by the ear canal bone wall. The transmitter coil 108 transmits electrical energy that includes power and electrical sound signals that includes the data, for example in a form of a wireless carrier signal, and data, for example in a form of a modulated wireless carrier signal, to the cochlear receiving coil 152. Power is transmitted between the two adjacent coils by electromagnetic induction.

Optionally, additional processor with another receiving coil (340, 212, respectively) is implanted below the skin preferably on the mastoid bone.

It is a challenge for a surgeon that implants the implanted portion of the hidden cochlear implant to position the receiving coil in an optimized position and place the external canal portion in an orientation that will assure efficiency and functionality of the hidden cochlear system, especially in view of the individual structure of the ear canal of each user.

### SUMMARY OF THE INVENTION

It is provided in accordance with an aspect of the present subject matter, devices that assists the surgeon or clinical expert to position the transmission coil within an ear canal unit or the receiving coil implanted within the mastoid cavity.

It is another aspect of the present subject matter to provide a method by which the surgeon can easily position the receiving coil during implementation.

It is therefore provided in accordance with an embodiment of the present subject matter, an in-canal positioning and orienting device is provided for assisting in placement of a receiving coil to be implanted within a human ear and/or a transmitting coil to be placed within an ear canal one in respect to the other, the in-canal positioning and orienting device comprising a transducer configured to collect feedback comprising features characterizing a positioning and orientation of the receiving coil to be implanted and the transmitting coil to be placed within the ear canal.

In accordance with another embodiment, the transducer is a transmitter and wherein at least one of the features is efficiency of transferring energy from the transmitter to the receiving coil in different orientations of the in-canal positioning and orienting device.

In accordance with another embodiment, the transmitter is a transmitting antenna.

In accordance with another embodiment, the transmitter is provided on an elongated member having an X axis along a length, and wherein the elongated member comprises an arm having an end onto which the transmitter is positioned, wherein the arm is configured to position and orient the transmitter within the ear canal, and wherein the elongated member further comprises an adjusting member configured to move the arm along the X axis and to tilt the arm in respect to the X axis so that the transmitter is configured to indicate a most effective placement of the transmitting coil that will be inserted to the ear canal.

In accordance with another embodiment, at least one of the features is an exact position of the transmitter in a space of the ear canal.

In accordance with another embodiment, the arm is replaceable. the transducer is a light transmitter and wherein at least one of the features is a position of the light transmitter relative to the fixed positioning of the transmitting coil.

In accordance with another embodiment, the light transmitter is a LED light.

In accordance with another embodiment, the LED light comprises different colors so that a color of the LED light can change as a result of the feedback.

In accordance with another embodiment, the device further comprises a microphone.

In accordance with another embodiment, the transducer is a transmitter coil and wherein the device is further comprises a LED.

In accordance with another embodiment, the receiving coil and the transmitting coil are parts of an in-canal wireless transmission system (ICWTS).

In accordance with yet another embodiment, it is provided a method of positioning and orienting an implanted receiving coil within a mastoid cavity in a vicinity of a transmitting coil placed in an ear canal of a human ear comprising:
implanting a receiving coil within the mastoid cavity in close proximity to the ear canal;
placing a transducer on an end of an elongated positioning tool;
inserting the transducer in the ear canal;
positioning and orienting the receiving coil while monitoring features characterizing relation between the receiving coil and the transducer;
recognizing a specific position and orientation in which a most effective power transmission between the receiving coil and the transmitting coil is anticipated; and
fixing a location of the receiving coil within the mastoid cavity.

The method of positioning and orienting an implanted receiving coil within a mastoid cavity as claimed in claim 13, wherein the transducer is a LED light.

In accordance with another embodiment, the method further comprising:
providing the LED light with at least two colors so that in a specific position and orientation of the receiving coil that is implanted, the color is changed.

In accordance with another embodiment, the method further comprising providing a transmitter besides the LED light.

It is also provided in accordance with yet an other embodiment a method of positioning and orienting an in-canal transmitting coil in the ear canal relative to an implanted receiving coil implanted within a mastoid cavity in a vicinity of the ear canal in a human ear comprising:
implanting a receiving coil within the mastoid cavity in close proximity to the ear canal and fixing the receiving coil in a specific location;
providing a transducer on an in-canal measurement tool wherein the transducer represents a positioning of the in-canal transmitting coil;
placing and orienting the transducer while monitoring features characterizing a relation between the receiving coil and the transducer;
recognizing a specific position and orientation in which a desired relation between the receiving coil and the transducer is achieved and detected; and
positioning and fixing the transmitting coil in the specific location in the ear canal.

In accordance with another embodiment, the transducer is a transmitter.

In accordance with another embodiment, the features are features selected from a group consisting of transmission efficiency, power transmitted, and data transmitted between the receiving coil and the transducer.

Unless otherwise defined, all technical and scientific terms used herein have the same meaning as commonly understood by one of ordinary skill in the art to which this disclosed subject matter belongs. Although methods and materials similar or equivalent to those described herein can be used in the practice or testing of the present disclosed subject matter, suitable methods and materials are described below. In case of conflict, the specification, including definitions, will control. In addition, the materials, methods, and examples are illustrative only and not intended to be limiting.

### BRIEF DESCRIPTION OF THE DRAWINGS

Some embodiments of the disclosed subject matter described, by way of example only, with reference to the accompanying drawings. With specific reference now to the drawings in detail, it is stressed that the particulars shown are by way of example and for purposes of illustrative discussion of the preferred embodiments of the present disclosed subject matter only, and are presented in the cause of providing what is believed to be the most useful and readily understood description of the principles and conceptual aspects of the disclosed subject matter. In this regard, no attempt is made to show structural details of the disclosed subject matter in more detail than is necessary for a fundamental understanding of the disclosed subject matter, the description taken with the drawings making apparent to those skilled in the art how the several forms of the disclosed subject matter may be embodied in practice.

In the drawings:

FIG. 1 illustrates a perspective view of a prior art hidden cochlear implanted system implanted within a human ear;

FIG. 2 illustrates positioning of the receiving coil within the human ear in a specific location, in accordance with some exemplary embodiments of the disclosed subject matter;

FIG. 3 illustrates a positioning device for accurate positioning of the canal unit within the ear canal, in accordance with some exemplary embodiments of the disclosed subject matter.

FIG. 4 illustrates an in-canal positioning tool with LED light for accurate positioning of the receiving coil during the surgery, in accordance with some exemplary embodiments of the disclosed subject matter; and

FIG. 5 illustrates an in-canal positioning tool provided with combination of LED and a transmission coil for accurate positioning of a receiving antenna during the surgery, in accordance with some exemplary embodiments of the disclosed subject matter.

### DETAILED DESCRIPTION OF THE INVENTION

Before explaining at least one embodiment of the disclosed subject matter in detail, it is to be understood that the disclosed subject matter is not limited in its application to the details of construction and the arrangement of the components set forth in the following description or illustrated in the drawings. The disclosed subject matter is capable of other embodiments or of being practiced or carried out in various ways. Also, it is to be understood that the phraseology and terminology employed herein is for the purpose of description and should not be regarded as limiting. The drawings are generally not to scale. For clarity, non-essential elements were omitted from some of the drawings.

The terms “comprises”, “comprising”, “includes”, “including”, and “having” together with their conjugates mean “including but not limited to”. The term “consisting of” has the same meaning as “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this disclosed subject matter may be presented in a range format. It should be understood that the description in range format is merely for convenience and brevity and should not be construed as an inflexible limitation on the scope of the disclosed subject matter. Accordingly, the description of a range should be considered to have specifically disclosed all the possible sub-ranges as well as individual numerical values within that range.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination or as suitable in any other described embodiment of the disclosed subject matter. Certain features described in the context of various embodiments are not to be considered essential features of those embodiments, unless the embodiment is inoperative without those elements.

The technology onto which an in-canal wireless transmission system (ICWTS) relies is electromagnetic induction of energy from a primary coil to a secondary coil. The induction of energy depends markedly on the electromagnetic field direction in the X, Y, Z plane of space and the angle of the coils relative to each other.

It is an aspect of the present subject matter to provide an in-canal positioning and orienting device for assisting in placement of a receiving coil to be implanted within a human ear and/or a transmitting coil to be placed within an ear canal one in respect to the other, the in-canal positioning and orienting device. The in-canal positioning and orienting device comprises a transducer configured to collect feedback comprising features characterizing a positioning and orientation of the receiving coil to be implanted and the transmitting coil to be placed within the ear canal.

At least two aspects are provided: according to one aspect, the receiving coil that is implanted within the mastoid bone in the vicinity the ear canal is being positioned and oriented relative to a position of the transmitting coil that will be placed within the ear canal. The transmitting coil is represented by a transducer that emits light that provides feedback on the relations between the transmitting and receiving coils. A second aspect is to implant the receiving coil in the mastoid bone and then use a transducer to find the best positioning of the transmitting coil relative to the receiving coil. The transducer in this case can be a transmitter that provides feedback on the efficiency of energy transfer from the transmitting coil to the receiving coil.

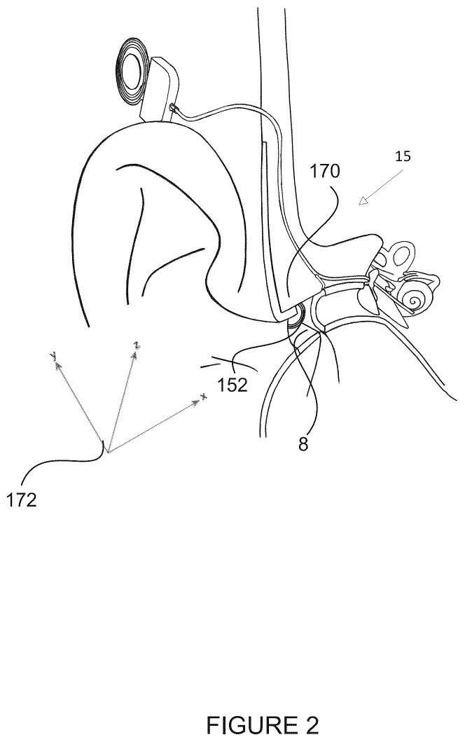

Reference is now made to FIG. 2 illustrating positioning of the receiving coil within the human ear in a specific location, in accordance with some exemplary embodiments of the disclosed subject matter.

It is important to note that the present subject matter aims at providing an ICWTS by which, in addition to the transfer of data between the external transmitting coil to the internal implanted receiving coil, energy in the form of power can be transferred as well. The transfer of energy from the external coil to the internal coil relies on electromagnetic induction. The efficiency of the induction of electrical current mainly depends on the positioning of the coils and their directions in the X, Y, Z planes in space as well as the angle of the coils relative to each other. A primary goal is to maintain the same distance, angle, and X, Y, Z position between the transmitting coil and the receiving coil given the variance in patient anatomy (only the implanted receiving coil can be seen in FIG. 2). A secondary goal is to allow easy positioning of the receiving coil while keeping the best efficiency as possible when the transmitting coil is inserted to within the canal. To do so, there is a need to allow the surgeon/clinical expert to easily change one of the coils position/orientation in the X, Y, Z axis.

Since, as mentioned hereinbefore, there is variance in patient anatomy that affects the positioning of the device in the ear, the external in canal portion as well as the internal implanted portion is to be specifically positioned. A primary goal for the ICWTS is to maintain a distance, angle, and X, Y, Z positioning of the receiving coil and the transmitting coil that will ensure the most effective power transmission.

A secondary goal of the present subject matter is to assist in easy positioning of the receiver coil while keeping the coils in an orientation in which the best efficiency is achieved. To do so, there is a need to allow a surgeon/clinical expert to be able to easily change one of the coils position and/or orientation in the X, Y, Z plane.

According to a first aspect of the present subject matter, it is provided a method of positioning the transmitting and receiving coils of the ICWTS in respect to a specific human ear. To achieve this goal, a receiving coil 152 of the implanted portion of the ICWTS is being positioned in a specific location during the implantation surgery. The optimized position for placing the receiving coil 152 of implanted unit 15 is within the mastoid bone 170 near the ear canal 8 adjacent and facing the ear canal inner wall. During the surgery, the surgeon uses dedicated software that shows results of features characterizing the relation between the coils such as transmission efficiency, power, data and more. this assists the surgeon to easily locate the transmission coil in its location in the ear canal. The surgeon that implants the implanted unit has to place the receiving coil 152 in a specific positioning while knowing that there is no additional option to move the implanted coil after implantation is completed while keeping the option to change the positioning of the transmitting coil of the external in canal unit (cannot be seen in FIG. 2) that is inserted to within the ear canal 8. Therefore, during surgery, the expert is able to determine the relations between the implanted receiving coil and the external in-canal transmitting coil, which position can be slightly changed by changing the transmission coil positioning in the canal while reading the software results until reaching the desire results.

In order to understand the directions within the ICWTS, a coordinate system 172 is shown in FIG. 2 while the X axis is directed in a direction that is parallel to the elongated axis of the ear canal. Receiving coil 152 is placed in a fixed positioning and distance in the Z axis direction from the ear canal 8. The Ear canal size creates a fixed position in the Y axis. One should bear in mind that the transmitting coil within the in-canal unit is allowed to move and change its orientation in the X axis direction and its orientation between the X-Y axes.

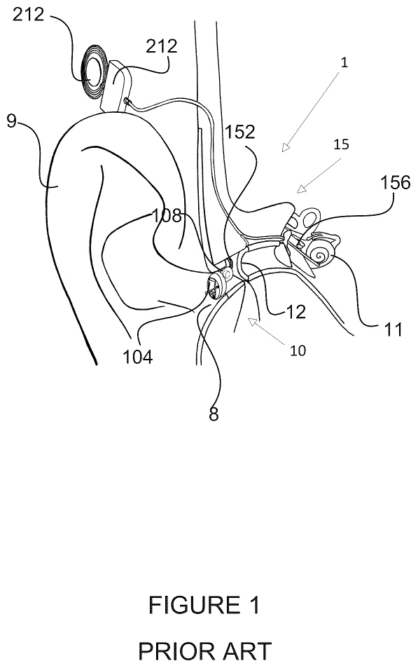

Returning to FIG. 1 in which the in-canal unit 10 can be seen in relation to the implanted unit 15. The transmitting coil 108 and the receiving coil 152 can be seen to be adjacent to one another while the ear canal wall separates them. Since the power is induced from the transmitting coil 108 to the receiving coil 152, the positioning of the coils has a significant role in the efficiency of the device.

According to an embodiment of the present subject matter, an in-canal measurements tool is provided, with specific mechanism that can provide the surgeon or clinical expert with appropriate information as feedback about the correct positioning of the transmission coil in respect to the receiving coil with the best efficiency in real time. The mechanism of the in-canal measurements tool can control the transmitting coil movement and orientation in the plane of the coordination system 172.

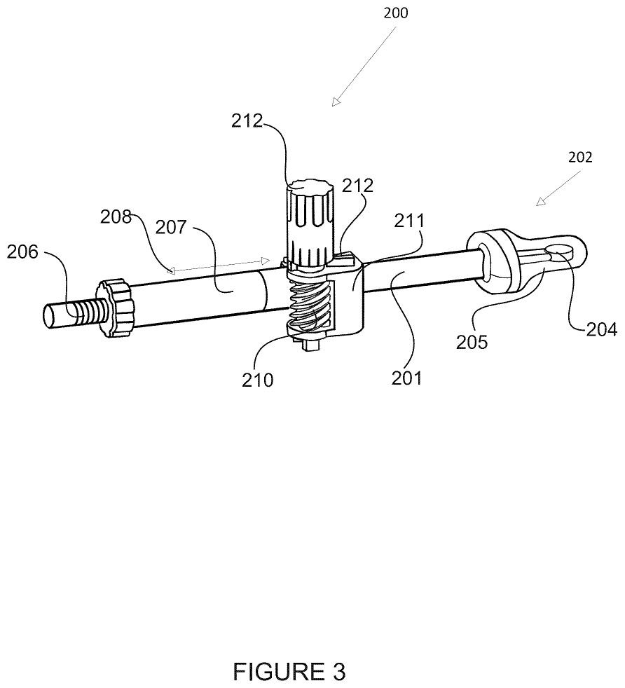

Reference is now made to FIG. 3 illustrating a positioning device for accurate positioning of the canal unit within the ear canal, in accordance with some exemplary embodiments of the disclosed subject matter.

The in-canal measurement tool 200, which is a positioning and orienting device, is an elongated device that comprises a measuring unit 202 at one side. Measuring unit 202 has a transmitter antenna 204 at its end that is covered by a protective cover 205. The measuring unit 202 is removably connected through an arm 201 to an elongated screw member 206 that is enveloped within a tube 207 that is configured to be elongated or shortened in an X direction as indicated by an arrow 208. Gear wheel 210 housed within an adjusting member 211 is mounted on and associated with elongated screw member 206 so that the movement of the gear wheel 210 assists in moving elongated screw member 206 in the X direction 208 using knob 212. Adjusting member 211 has a tilting element 212 that is configured to tilt the elongated screw member 206 in an angle relative to the X axis parallel to arrow 208.

According to a primary method of the present subject matter, the following steps are provided: locating the receiving coil of the implanted unit in a specific desired location and then, using the in-canal measurement tool 200 to locate the transmitting coil in the best position relative to the implanted receiving coil. Using the in-canal measurement tool 200, the distance between the receiving coil and the transmitting coil is determined as well as their orientation and then, the final position of the transmitter coil is determined and fixed.

As mentioned herein before, the in-canal measurement tool 200 comprises transmitter antenna 204 wherein the transmitter antenna has the same features as the transmitting coil of the in canal unit of the ICWTS. The transmitter antenna is configured to transfer signals relating to its relation with the implanted receiving coil to the surgeon. Accordingly, the surgeon can determine the relations between the coils and the result of the positioning using one of the following methods in which the positioning device is inserted to within the canal in the ear:

According to one example, determining the distance and orientation between the implanted receiving coil and the in-canal transmitting coil is performed by transmitting short pulses using transmitter antenna 204 and measuring the rate of decay of the pulses received by the implanted receiving coil (152 in FIGS. 1 and 2). As the distance between the transmitter antenna 204 and the receiving coil is shorter, the rate of decay of the pulses is higher. Thus, a correlation between the decay rate and the distance between the antenna and the receiving coil can be determined or calculated. The results are used to determine the most effective distance between the transmitting antenna and the receiving coil. It should be noted that the transmitter antenna 204 can be moved using the adjusting member 211 by which the antenna can be moved in the X direction and can be tilted in an angle relative to the X direction. The positioning of the transmitter antenna 204 can then be determined.

Another example for the method to determine an effective positioning of the coils is to let the transmitter antenna 204 in the canal to transmit signals in various different frequencies, and determine the frequency that produces the highest voltage in the implanted receiving coil. This frequency producing the highest voltage is the common resonance frequency of the antenna and the coil. The common resonance frequency depends on the distance between the two. Thus, a correlation between the resonance frequency and the distance can be determined and used for determining the distance between the antenna and the receiving coil.

Another example for the method to determine an effective positioning of the coils is to calculate the power transmission efficiency between the coils and keep changing the transmitter coil positions. This is performed until the relevant efficiency is found.

Accordingly, the surgeon can determine the final positioning of the implanted receiving coil and the transmitter antenna that represents the positioning of the transmitting coil in the in-canal unit that will be inserted.

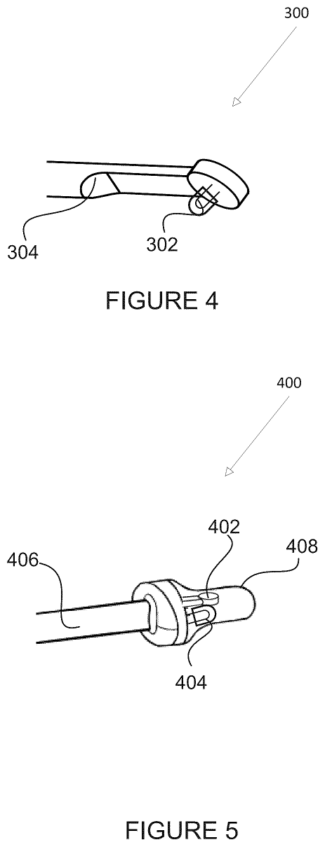

Reference is now made to FIG. 4 illustrating an in-canal positioning tool with LED light for accurate positioning of the receiving coil during the surgery, in accordance with some exemplary embodiments of the disclosed subject matter.

According to a second aspect of the present subject matter, it is provided a method of positioning the transmitting and receiving coils of the ICWTS in respect to a specific human ear. To achieve this goal, a receiving coil 152 of the implanted portion of the ICWTS is being positioned according to the transmission unit positioning during the implantation surgery. During the surgery, the surgeon uses dedicated software that shows results of features characterizing the relation between the coils such as transmission efficiency, power, data etc. to easily locate the receiving coil in its location in the mastoid cavity in the user's head according to the transmission unit position (in-canal positioning tool).

According to the method that is described herein, an in-canal positioning tool 300 is provided with a LED light 302 at an end of an arm 304. The LED light is strong enough to be seen by the surgeon from his respective view from inside the mastoid cavity, where he is implanting the receiving coil. Using an arm with a LED light provides the surgeon with an option to visually position the receiving coil (the coil is not seen in this figure) in place during the surgery itself according to the patient specific ear structure.

Optionally or additionally, arm 304 can be incorporated within the in-canal measurement tool 200 instead of arm 201 shown in FIG. 3.

Optionally or additionally, in-canal measurement tool 200 can be provided with a transmitter antenna as shown in FIG. 3. This combination of LED light 302 and transmitter coil can provide the surgeon both visual confirmation and a data based validation of the relations between the receiving coil and the transmitting coil that is provided in the in-canal unit.

Optionally or additionally, in-canal positioning tool is provided with a plurality of LED lights. The different LED light has different colors so that when the surgeon finds the best location and orientation of the receiving coil, the color of the LED light turned into another color. This can assist the surgeon to fix the receiving coil in a certain positioning.

Optionally or additionally, in-canal positioning tool 300 is provided with electrocochleography system. Accordingly, a microphone is provided that sends sound data directly to the ear drum in order to create stimulation in the cochlea nerve and help testing feedback of the natural organs to the cochlear implant provided with the receiving coil.

Reference is now made to FIG. 5 illustrating an in-canal positioning tool with combination of LED and a transmission coil for accurate positioning of a receiving coil during surgery, in accordance with some exemplary embodiments of the disclosed subject matter.

According to the method that is described herein, an in-canal positioning tool 400 is provided with a transmitter antenna 402 and a LED light 404 at an end of an arm 406. The LED light is strong enough to be seen by the surgeon during the implantation of the receiving coil while at the same time, determination of the most effective distance between the implanted receiving coil and the transmitter antenna 404 is performed according to the methods discussed hereinbefore. Both elements are covered by a protecting cover that can be manufactured specifically to the human ear canal dimension 408 of a specific user. The arm 406 carrying the positioning tool is moved to within the ear canal during surgery and can be moved in the X-direction as indicated hereinbefore.

The following are additional examples of methods by which the in-canal tool or positioning device can be used for positioning and orientation purposes, while the choice of method depends on the required precision, range, and application:

According to one example, the positioning device comprises an inertial measurement unit such as gyroscope, accelerometer, or magnetometer. A gyroscope can be located parallel and close to the transmitting coil and the receiving coil. Small changes in the gyroscope will determine the angle of the coils in the X axis dimension. Small changes in the accelerometer and magnetometer can be used to measure the X and Y positions of the coils. This information can be used by the surgeon during surgery in 3D.

Another method of measuring distance in specific axis is using optical methods such as laser displacement sensors. The use of laser beams to measure distance is based on the reflection from the object's surface. This can provide precision manufacturing, robotics, and surface profiling. This method can render high accuracy and resolution.

Another method uses optical encoders. Light sources and photo detectors are used to measure the movement of an encoded disk or strip to measure displacement by comparing the phase difference of two light beams.

Mechanical methods such as linear potentiometers can also be used. Displacement is measured through change in resistance such as a slider that moves along a track.

Inductive methods can be used also through using inductive proximity sensors that measure the change in inductance caused by the presence of a metallic object.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

Another example is to calculate the power transmission efficiency between the coils and keep changing the positions until finding the relevant efficiency.

1. An in-canal positioning and orienting device for assisting in placement of a receiving coil to be implanted within a human ear and/or a transmitting coil to be placed within an ear canal one in respect to the other, the in-canal positioning and orienting device comprising a transducer configured to collect feedback comprising features characterizing a positioning and orientation of the receiving coil to be implanted and the transmitting coil to be placed within the ear canal.

2. The in-canal positioning and orienting device as claimed in claim 1, wherein for assisting in placement of the transmitting coil in respect to a fixed positioning of the receiving coil, the transducer is a transmitter and wherein at least one of the features is an efficiency of transferring energy from the transmitter to the receiving coil in different orientations of the in-canal positioning and orienting device.

3. The in-canal positioning and orienting device as claimed in claim 2, wherein the transmitter is a transmitting antenna.

4. The in-canal positioning and orienting device as claimed in claim 2, wherein the transmitter is provided on an elongated member having an X axis along a length, and wherein the elongated member comprises an arm having an end onto which the transmitter is positioned, wherein the arm is configured to position and orient the transmitter within the ear canal, and wherein the elongated member further comprises an adjusting member configured to move the arm along the X axis and to tilt the arm in respect to the X axis so that the transmitter is configured to indicate a most effective placement of the transmitting coil that will be inserted to the ear canal.

5. The in-canal positioning and orienting device as claimed in claim 4, wherein at least one of the features is an exact position of the transmitter in a space of the ear canal.

6. The in-canal positioning and orienting device as claimed in claim 4, wherein the arm is replaceable.

7. The in-canal positioning and orienting device as claimed in claim 1, for assisting in placement of the receiving coil in respect to a fixed positioning of the transmitting coil, the transducer is a light transmitter and wherein at least one of the features is a position of the light transmitter relative to the fixed positioning of the transmitting coil.

8. The in-canal positioning and orienting device as claimed in claim 7, wherein the light transmitter is a LED light.

9. The in-canal positioning and orienting device as claimed in claim 8, wherein the LED light comprises different colors so that a color of the LED light can change as a result of the feedback.

10. The in-canal positioning and orienting device as claimed in claim 1, wherein the device further comprises a microphone.

11. The in-canal positioning and orienting device as claimed in claim 1, wherein the transducer is a transmitter coil and wherein the device is further comprises a LED.

12. The in-canal positioning and orienting device as claimed in claim 1, wherein the receiving coil and the transmitting coil are parts of an in-canal wireless transmission system (ICWTS).

13. A method of positioning and orienting an implanted receiving coil within a mastoid cavity in a vicinity of a transmitting coil placed in an ear canal of a human ear comprising:
implanting a receiving coil within the mastoid cavity in close proximity to the ear canal;
placing a transducer on an end of an elongated positioning tool;
inserting the transducer in the ear canal;
positioning and orienting the receiving coil while monitoring features characterizing relation between the receiving coil and the transducer;
recognizing a specific position and orientation in which a most effective power transmission between the receiving coil and the transmitting coil is anticipated; and
fixing a location of the receiving coil within the mastoid cavity.

14. The method of positioning and orienting an implanted receiving coil within a mastoid cavity as claimed in claim 13, wherein the transducer is a LED light.

15. The method of positioning and orienting an implanted receiving coil within a mastoid cavity as claimed in claim 13, wherein the method further comprising:
providing the LED light with at least two colors so that in a specific position and orientation of the receiving coil that is implanted, the color is changed.

16. The method of positioning and orienting an implanted receiving coil within a mastoid cavity as claimed in claim 14, wherein the method further comprising providing a transmitter besides the LED light.

17. A method of positioning and orienting an in-canal transmitting coil in the ear canal relative to an implanted receiving coil implanted within a mastoid cavity in a vicinity of the ear canal in a human ear comprising:
implanting a receiving coil within the mastoid cavity in close proximity to the ear canal and fixing the receiving coil in a specific location;
providing a transducer on an in-canal measurement tool wherein the transducer represents a positioning of the in-canal transmitting coil;
placing and orienting the transducer while monitoring features characterizing a relation between the receiving coil and the transducer;
recognizing a specific position and orientation in which a desired relation between the receiving coil and the transducer is achieved and detected; and
positioning and fixing the transmitting coil in the specific location in the ear canal.

18. The method of positioning and orienting an in-canal transmitting coil in the ear canal as claimed in claim 17, wherein the transducer is a transmitter.

19. The method of positioning and orienting an in-canal transmitting coil in the ear canal as claimed in claim 17, wherein the features are features selected from a group consisting of transmission efficiency, power transmitted, and data transmitted between the receiving coil and the transducer.
